(19) United States
(12) Patent Application Publication
(10) Pub. No.: US 2026/0118524 A1
(43) Pub. Date: Apr. 30, 2026

(54) LIGHT DETECTION DEVICE

(71) Applicant: InnoCare Optoelectronics Corporation, Tainan City (TW)

(72) Inventors: Shih-Hsien Yang, Tainan City (TW); Sung-Pao Cheng, Tainan City (TW)

(73) Assignee: InnoCare Optoelectronics Corporation, Tainan City (TW)

(21) Appl. No.: 19/000,621

(22) Filed: Dec. 23, 2024

(30) Foreign Application Priority Data
Mar. 14, 2024 (TW) 113109393

(51) Int. Cl. G01T 1/02

(52) U.S. Cl. CPC G01T 1/02

(57) ABSTRACT

A light detection device is provided. The light detection device includes a detection panel, first gate driving circuits, a second gate driving circuit, and a controller. The detection panel includes first areas, at least one second area, first scan line groups, and at least one second scan line. The controller controls the second gate driving circuit during a first period, and detect a dose of the input light by using charges generated by the at least one second area. The controller controls the first gate driving circuits and the second gate driving circuit during a second period, and generate a data image by using charges generated by the first areas and the at least one second area.

### CROSS-REFERENCE TO RELATED APPLICATION

This application claims the priority benefit of Taiwan application serial no. 113109393, filed on Mar. 14, 2024. The entirety of the above-mentioned patent application is hereby incorporated by reference herein and made a part of this specification.

### BACKGROUND

### Technical Field

The disclosure relates to a light detection device, and more particularly, to a light detection device with an automatic exposure detection (AED) function.

### Description of Related Art

Generally speaking, a light detection device receives input light (e.g., X-rays, etc.) provided by a light source device, and generates a data image based on a dose of the input light. The light detection device may determine whether a light source outputs the input light by using an automatic exposure detection (AED) function. However, the current AED is to sequentially scan all pixel rows of the light detection device to determine whether the input light is provided by using all scan lines of the light detection device. Therefore, the current AED is quite time-consuming, which greatly reduces the time it takes for the light detection device to generate the data image. As a result, how to shorten the operation time for AED is one of the research focuses for those skilled in the art.

### SUMMARY

The disclosure provides a light detection device, which may shorten operation time for automatic exposure detection (AED).

In an embodiment of the disclosure, a light detection device includes a detection panel, multiple first gate driving circuits, a second gate driving circuit, and a controller. The detection panel converts input light into a charge. The detection panel includes multiple first areas, at least one second area, multiple first scan line groups, and at least one second scan line. The first areas are correspondingly coupled to the first scan line groups. The at least one second area is correspondingly coupled to the at least one second scan line. The first gate driving circuits are correspondingly coupled to the first scan line groups. The second gate driving circuit is coupled to the at least one second scan line. The controller is coupled to the first gate driving circuits and the second gate driving circuit. The controller controls the second gate driving circuit during a first period, and detects a dose of the input light by using a charge generated by the at least one second area. The controller controls the first gate driving circuits and the second gate driving circuit during a second period, and generates a data image by using a charge generated by the first areas and the at least one second area.

Based on the above, the light detection device detects the dose of the input light by using the charge generated by the at least one second area. In this way, the operation time for AED may be shortened.

### BRIEF DESCRIPTION OF THE DRAWINGS

FIG. 1 is a schematic view of a light detection device according to the first embodiment of the disclosure.

FIG. 2 is a schematic view of a dose according to an embodiment of the disclosure.

FIG. 3 is a schematic view of a light detection device according to the second embodiment of the disclosure.

FIG. 4 is a schematic view of a light detection device according to the third embodiment of the disclosure.

### DETAILED DESCRIPTION OF DISCLOSED EMBODIMENTS

Some embodiments of the disclosure will be described in detail with reference to the accompanying drawings. For reference numerals cited in the following descriptions, the same reference numerals appearing in different drawings are regarded as the same or similar components. The embodiments are only a part of the disclosure and do not disclose all possible implementations of the disclosure. More precisely, the embodiments are merely examples of the device and the method.

Throughout the disclosure and the appended claims, certain words are used to refer to specific components. Those skilled in the art should understand that electronic device manufacturers may refer to the same components by different names. The disclosure does not intend to distinguish those components with the same function but different names. In the following description and claims, the terms “contain” and “include” are open-ended terms, so they should be interpreted as “include but not limited to . . . ”.

Direction terms mentioned in this specification, such as such as “up,” “down,” “front,” “back,” “left,” and “right,” merely refer to directions in the accompanying drawings. Therefore, the direction terms used is for illustration, not for limiting this disclosure. In the drawings, each drawing shows the general features of the method, structure, and/or material used in a specific embodiment. However, these drawings should not be construed as defining or limiting the scope or nature of the embodiments. For example, for the sake of clarity, the relative size, thickness, and position of each layer, region, and/or structure may be reduced or enlarged.

In some embodiments of the disclosure, terms such as “bonded”, “connected”, “interconnected”, etc. regarding bonding and connection, unless specifically defined, can mean that two structures are in direct contact, or that two structures are not directly in contact, where there are other structures located between the two structures. Moreover, the terms of joining and connecting can also include the case where both structures are movable or both structures are fixed. In addition, the word “coupled” may include to any direct or indirect electrical connection means. In the case of direct electrical connection, the terminals of the elements on the two circuits are directly connected or connected to each other by a conductor line segment. In the case of indirect electrical connection, a switch, diode, capacitor, inductor, resistor, other suitable elements, or a combination of the above components is provided between the terminals of the elements on the two circuits, but the disclosure is not limited thereto.

The terms “about”, “equal to”, “identical” or “same”, “substantially”, or “approximately” are generally interpreted as being within 20% of a given value or range or are interpreted as being within 10%, 5%, 3%, 2%, 1%, or 0.5% of a given value or range.

The ordinal numbers used in the specification and claims, such as “first”, “second”, etc., are used to modify the elements, and they do not imply or represent the (or these) elements have any previous ordinal numbers, do not represent the order of a element and another element, or the order of a manufacturing method. The use of these ordinal numbers is only used to clearly distinguish an element with a certain name from another element with the same name. The terms used in the claims and the specification may not have to be the same, and accordingly, the first component provided in the specification may be the second component in the claims. It should be understood that in the following embodiments, the technical features of several different embodiments may be replaced, recombined, and mixed to complete other embodiments without departing from the spirit of the disclosure.

It should be understood that in the following embodiments, the features of several different embodiments may be replaced, recombined, and mixed to complete other embodiments without departing from the spirit of the disclosure. As long as the features of the embodiments do not violate or do not conflict with the spirit of the disclosure, they may be mixed and matched arbitrarily.

Unless otherwise defined, all terms (including technical and scientific terms) used herein have the same meaning as commonly understood by a person of ordinary skill in the art. It will be further understood these terms, such as those defined in commonly used dictionaries, should be interpreted as having meaning that is consistent with their meaning in the context of the related art and the disclosure and should not be interpreted in an idealized or overly formal sense unless expressly so defined herein.

The electronic device in the disclosure may include a light detection device or a splicing device, but the disclosure is not limited thereto. The electronic device (e.g., a light detector) may be a bendable or flexible electronic device. In the disclosure, the electronic device (e.g., a light detector) may include an electronic element, and the electronic element may include a passive element and an active element, such as a capacitor, a resistor, an inductor, a diode, a transistor, etc. The diode may include a light emitting diode or a photodiode. The light emitting diode may include, for example, an organic light emitting diode (OLED), a mini light emitting diode (mini LED), a micro light emitting diode (micro LED), or a quantum dot light emitting diode (quantum dot LED), but the disclosure is not limited thereto. Hereinafter, the detection device will be used as the electronic device or the splicing device to illustrate the disclosure, but the disclosure is not limited thereto.

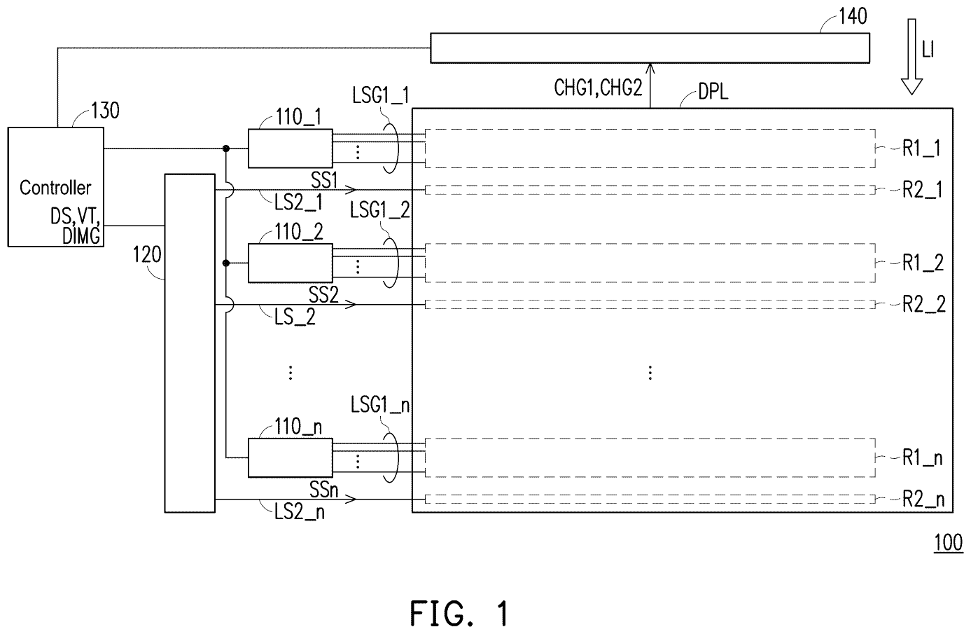

Referring to FIG. 1, FIG. 1 is a schematic view of a light detection device according to the first embodiment of the disclosure. In this embodiment, a light detection device 100 includes a detection panel DPL, first gate driving circuits 110_1 to 110_n, a second gate driving circuit 120, and a controller 130. The detection panel DPL may convert input light LI into a charge (e.g., a charge CHG1 or a charge CHG2). For example, the input light LI may be an X-ray, but the disclosure is not limited thereto. The detection panel DPL may convert the input light LI into visible light, and convert the visible light into the charge.

The detection panel DPL includes first areas R1_1 to R1_n, second areas R2_1 to R2_n, first scan line groups LSG1_1 to LSG1_n, and second scan lines LS2_1 to LS2_n. Each of the first scan line groups LSG1_1 to LSG1_n includes multiple first scan lines. The first areas R1_1 to R1_n are coupled correspondingly to the first scan line groups LSG1_1 to LSG1_n. The second areas R2_1 to R2_n are coupled correspondingly to the second scan lines LS2_1 to LS2_n.

Taking this embodiment as an example, the first area R1_1 is coupled to the first scan line group LSG1_1. The first area R1_2 is coupled to the first scan line group LSG1_2, and the rest may be derived by analogy. The second area R2_1 is coupled to the second scan line LS2_1. The second area R2_2 is coupled to the second scan line LS2_2, and the rest may be derived by analogy.

In this embodiment, the first gate driving circuits 110_1 to 110_n are coupled correspondingly to the first scan line groups LSG1_1 to LSG1_n. Taking this embodiment as an example, each of the first scan line groups LSG1_1 to LSG1_n includes the first scan lines. The first gate driving circuit 110_1 is coupled to the first scan lines of the first scan line group LSG1_1. The first gate driving circuit 110_2 is coupled to the first scan lines of the first scan line group LSG1_2, and the rest may be derived by analogy. The second gate driving circuit 120 is coupled to the second scan lines LS2_1 to LS2_n.

In this embodiment, the controller 130 is coupled to the first gate driving circuits 110_1 to 110_n and the second gate driving circuit 120. The controller 130 controls the second gate driving circuit 120 during a first period, and detects a dose DS of the input light LI by using the charge CHG1 (i.e., a first period charge) generated by at least one of the second areas R2_1 to R2_n. In addition, the controller 130 controls the first gate driving circuits 110_1 to 110_n and the second gate driving circuit 120 during a second period. The controller 130 generates a data image DIMG by using the charge CHG2 (i.e., a second period charge) generated by the first areas R1_1 to R1_n and the second areas R2_1 to R2_n.

Generally speaking, current automatic exposure detection (AED) is to sequentially scan all pixel rows of the light detection device to determine whether the input light LI is provided by using all scan lines of the light detection device. It is worth mentioning here that the light detection device 100 in this embodiment may detect the dose DS of the input light LI by using the charge CHG1 generated by the second areas R2_1 to R2_n. The light detection device 100 may not be required to sequentially scan all pixel rows of the light detection device 100 to determine whether the input light LI is provided by using all the scan lines. In this way, operation time for AED of the detection device 100 may be shortened.

In this embodiment, the light detection device 100 further includes a readout circuit 140. The readout circuit 140 is coupled to the first areas R1_1 to R1_n, the second areas R2_1 to R2_n, and the controller 130. The readout circuit 140 provides the charge (i.e., the charge CHG1 or the charge CHG2) generated by at least one of the first areas R1_1 to R1_n and the second areas R2_1 to R2_n to the controller 130.

In this embodiment, the first areas R1_1 to R1_n are separated by the second areas R2_1 to R2_n. Furthermore, the number of first areas R1_1 to R1_n is equal to the number of second areas R2_1 to R2_n, and the first areas R1_1 to R1_n and the second areas R2_1 to R2_n are arranged alternately. However, the disclosure is not limited thereto. In some embodiments, the number of second areas may be one (i.e., one of the second areas R2_1 to R2_n). For example, the light detection device 100 only includes the single second area R2_1. The second area R2_1 separates the first areas R1_1 and R1_2.

In this embodiment, the second areas R2_1 to R2_n are coupled to the second scan lines LS2_1 to LS2_n in a one-to-one manner, but the disclosure is not limited thereto. In some embodiments, the second area R2_1 is coupled to multiple second scan lines.

In this embodiment, the controller 130 is, for example, a central processing unit (CPU), or other programmable general-purpose or special-purpose microprocessors, digital signal processors (DSP), programmable controllers, application specific integrated circuits (ASIC), programmable logic devices (PLD), or other similar devices or a combination of these devices, which may load and execute a computer program. In this embodiment, the first gate driving circuits 110_1 to 110_n and the second gate driving circuit 120 include shift registers respectively.

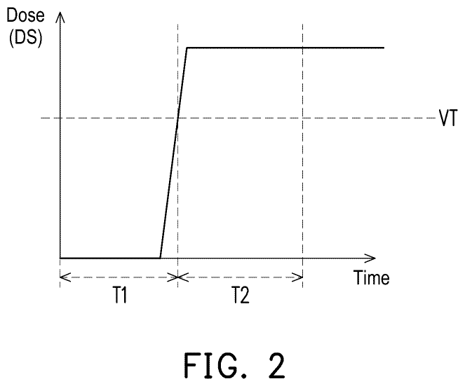

Referring to FIG. 1 and FIG. 2, FIG. 2 is a schematic view of a dose according to an embodiment of the disclosure. In this embodiment, during a first period T1, the light detection device 100 discharges the charge of the first areas R1_1 to R1_n. For example, the controller 130 may control the first gate driving circuits 110_1 to 110_n to discharge the charge of the first areas R1_1 to R1_n during the first period T1. Therefore, during the first period T1, the first areas R1_1 to R1_n do not provide the charge CHG1.

During the first period T1, the controller 130 controls the second gate driving circuit 120. The second gate driving circuit 120 provides scan signals SS1 to SSn to the second scan lines LS2_1 to LS2_n at the same time. For example, the second scan line LS2_1 receives the scan signal SS1. The second scan line LS2_2 receives the scan signal SS2, and the rest may be derived by analogy. The scan signals SS1 to SSn have the same timing. The second areas R2_1 to R2_n are driven at the same time. Therefore, the charge CHG1 corresponding to the second areas R2_1 to R2_n may be provided at the same time.

The controller 130 receives the charge CHG1 during the first period T1. During the first period T1, the charge CHG1 of the input light LI is positively correlated with the dose DS. Therefore, the controller 130 may obtain the dose DS according to the charge CHG1. When the dose DS is less than a critical value VT, it means that intensity of the input light LI received by the detection panel DPL is insufficient. For example, the input light LI may not have been provided. Therefore, the controller 130 maintains an operation during the first period T1, thereby continuing to detect the dose DS by using the charge CHG1 of the second areas R2_1 to R2_n.

During the first period T1, when the dose DS is greater than or equal to the critical value VT, it means that the intensity of the input light LI received by the detection panel DPL is sufficient. For example, the input light LI is provided. The controller 130 controls the first gate driving circuits 110_1 to 110_n and the second gate driving circuit 120 during a second period T2. The controller 130 controls the first gate driving circuits 110_1 to 110_n to stop discharging the charge of the first areas R1_1 to R1_n during the second period T2. Therefore, the first areas R1_1 to R1_n and the second areas R2_1 to R2_n generate the charge CHG2 during the second period T2.

In this embodiment, during the second period T2, the controller 130 controls the first gate driving circuits 110_1 to 110_n and the second gate driving circuit 120. The first gate driving circuits 110_1 to 110_n respectively provide scan signals to multiple scan lines of the corresponding first scan line group. The second gate driving circuit 120 sequentially provides the scan signals SS1 to SSn to the second scan lines LS2_1 to LS2_n during the second period T2. Therefore, multiple scan lines of the first scan line groups LSG1_1 to LSG1_n and the second scan lines LS2_1 to LS2_n sequentially receive scan signals of different timings in an order of arrangement directions of the first areas R1_1 to R1_n and the second areas R2_1 to R2_n. In other words, the scan lines of the first scan line groups LSG1_1 to LSG1_n and the second scan lines LS2_1 to LS2_n may be driven sequentially based on the above arrangement directions. For example, the charge CHG2 is generated in multiple pixel rows of first area R1_1. Then, the charge CHG2 is generated in at least one pixel row of the second area R2_1. Next, the charge CHG2 is generated in multiple pixel rows of the first area R1_2, and the rest may be derived by analogy.

For example, each of the first gate driving circuits 110_1 to 110_n includes 512 pins (but the disclosure is not limited thereto). The first pin to the 511th pin of each of the first gate driving circuits 110_1 to 110_n are electrically connected to corresponding different scan lines. The first scan line group LSG1_1 includes the first scan line to the 511th scan line. The first pin of the first gate driving circuit 110_1 is electrically connected to the first scan line. The second pin of the first gate driving circuit 110_1 is electrically connected to the second scan line, and the rest may be derived by analogy. The 512th pin of the first gate driving circuit 110_1 is not connected to the scan line. The second scan line LS2_1 connected to the second gate driving circuit 120 is the 512th scan line. The first scan line group LSG1_2 includes the 513th scan line to the 1023rd scan line. The first pin of the first gate driving circuit 110_2 is electrically connected to the 513th scan line. The second pin of the first gate driving circuit 110_2 is electrically connected to the 514th scan line, and the rest may be derived by analogy. The 512th pin of the first gate driving circuit 110_2 is not connected to the scan line. The second scan line LS2_2 connected to the second gate driving circuit 120 is the 1024th scan line. During the second period T2, all the first scan lines and second scan lines LS2_1 to LS2_n are scanned based on the above arrangement directions. In other words, the first scan line to the second scan line LS2_n may be driven sequentially based on the above arrangement directions. In addition, a time difference between the sequential driving of the two adjacent scan lines of the first scan line to the second scan line LS2_n is, for example, the same (but the disclosure is not limited thereto).

In addition, the controller 130 generates the data image DIMG according to the charge CHG2 during the second period T2. The controller 130 compensates partial images corresponding to the second areas R2_1 to R2_n.

In this embodiment, the partial images corresponding to the second areas R2_1 to R2_n in the data image DIMG may have weaker or stronger gray levels. Therefore, in order to enable the data image DIMG to have better display quality, the controller 130 may compensate the gray levels of the partial images corresponding to the second areas R2_1 to R2_n. In this embodiment, the partial images corresponding to the second areas R2_1 to R2_n may be compensated by using an image compensation method well known to those skilled in the art. For example, the controller 130 may compensate the partial image of the second area R2_1 according to the partial images of the first areas R1_1 and R1_2 adjacent to the second area R2_1. The controller 130 may compensate the partial image of the second area R2_2 according to the partial images of the first areas R1_2 and R1_3 adjacent to the second area R2_2, and the rest may be derived by analogy.

In some embodiments, during the first period T1, the second gate driving circuit 120 sequentially provides the scan signals SS1 to SSn to the second scan lines LS2_1 to LS2_n. For example, the second scan line LS2_1 receives the scan signal SS1. The second scan line LS2_2 receives the scan signal SS2, and the rest may be derived by analogy. The timing of the scan signal SS2 lags behind the timing of the scan signal SS1. Therefore, the second scan lines LS2_1 to LS2_n sequentially receives the scan signals SS1 to SSn of different timings in the order of the arrangement directions of the first areas R1_1 to R1_n and the second areas R2_1 to R2_n. The second areas R2_1 to R2_n are driven sequentially. The charges CHG1 corresponding to the second areas R2_1 to R2_n may be provided sequentially. In this embodiment, when the dose DS is greater than or equal to the critical value VT, the controller 130 generates the data image DIMG according to the charge CHG2 during the second period T2.

It should be noted that in this embodiment, during the first period T1, the charges CHG1 corresponding to the second areas R2_1 to R2_n may be provided sequentially. The partial image corresponding to one of the second areas R2_1 to R2_n in the data image DIMG may have the weaker or stronger gray level. Therefore, for example, during the first period T1, when the dose DS of the charge CHG2 corresponding to the second area R2_2 is greater than or equal to the critical value VT, the partial image corresponding to the second area R2_2 may have the weaker or stronger gray level. Therefore, the controller 130 may compensate the partial image of the second area R2_2 according to the partial images of the first areas R1_2 and R1_3 adjacent to the second area R2_2.

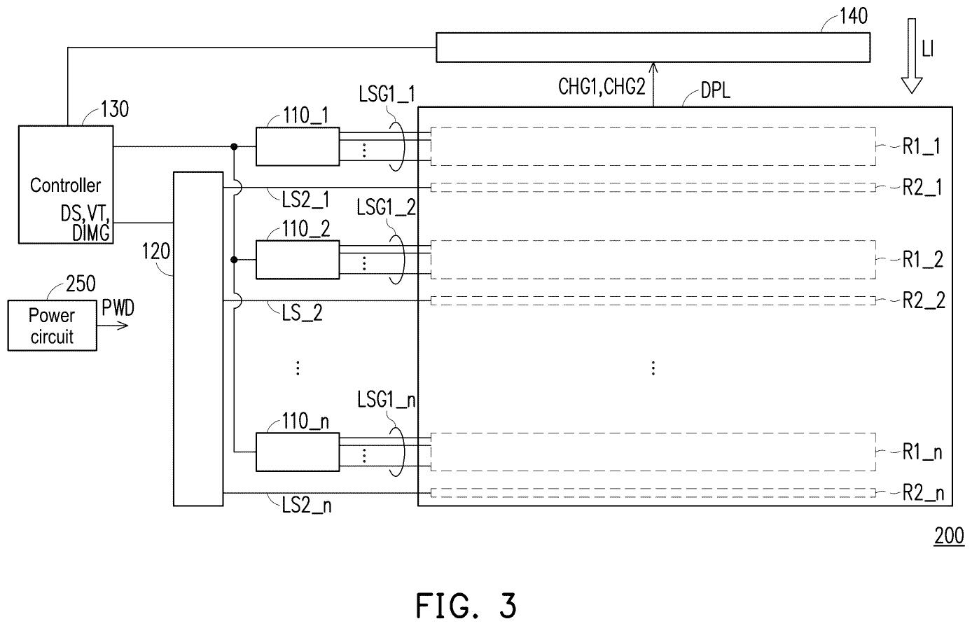

Referring to FIG. 3, FIG. 3 is a schematic view of a light detection device according to the second embodiment of the disclosure. In this embodiment, a light detection device 200 includes the detection panel DPL, the first gate driving circuits 110_1 to 110_n, the second gate driving circuit 120, the controller 130, the readout circuit 140, and a power circuit 250. Implementation details of the detection panel DPL, the first gate driving circuits 110_1 to 110_n, the second gate driving circuit 120, the controller 130, and the readout circuit 140 have been clearly described in the embodiments of FIG. 1 and FIG. 2. Therefore, the same details will not be repeated in the following.

In this embodiment, the power circuit 250 generates a driving power PWD. For example, the driving power PWD includes a voltage source or a current source configured to drive the detection panel DPL, the first gate driving circuits 110_1 to 110_n, the second gate driving circuit 120, the controller 130, and the readout circuit 140.

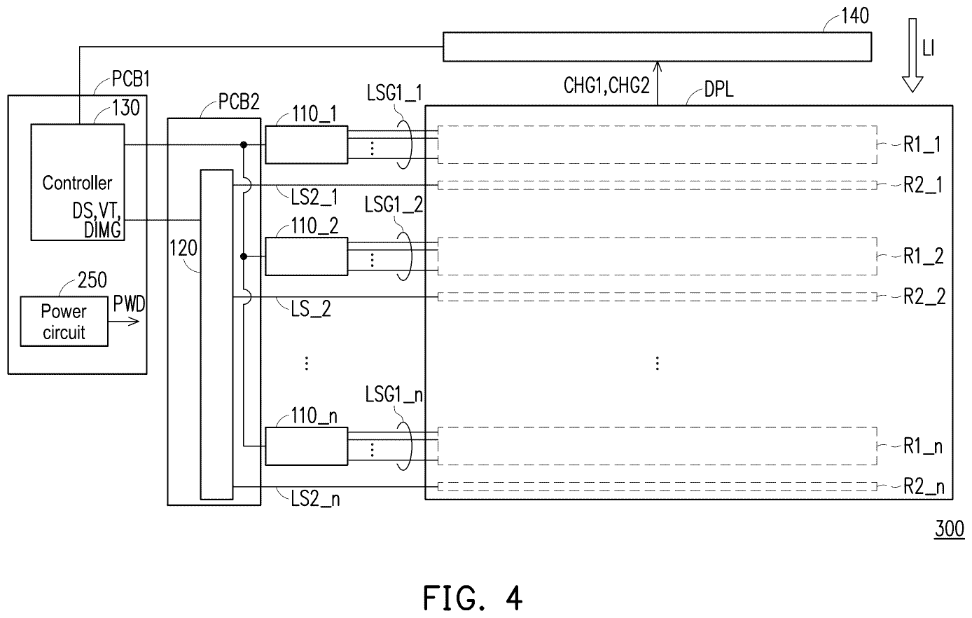

Referring to FIG. 4, FIG. 4 is a schematic view of a light detection device according to the third embodiment of the disclosure. In this embodiment, the light detection device 300 includes the detection panel DPL, the first gate driving circuits 110_1 to 110_n, the second gate driving circuit 120, the controller 130, the readout circuit 140, the power circuit 250, and circuit boards PCB1 and PCB2. The implementation details of the detection panel DPL, the first gate driving circuits 110_1 to 110_n, the second gate driving circuit 120, the controller 130, and the readout circuit 140 have been clearly described in the embodiments of FIG. 1 and FIG. 2. Therefore, the same details will not be repeated in the following. Implementation details of the power circuit 250 have been clearly described in the embodiment of FIG. 3. Therefore, the same details will not be repeated in the following.

In this embodiment, the controller 130 and the power circuit 250 are disposed on the circuit board PCB1. The second gate driving circuit 120 is disposed on the circuit board PCB2. For example, the circuit boards PCB1 and PCB2 may be implemented by a rigid substrate or a flexible substrate respectively. The rigid substrate may be implemented by a bakelite plate, fiberglass, or various plastic plates, but the disclosure is not limited thereto. The flexible substrate may be implemented by a soft plastic substrate, but the disclosure is not limited thereto.

In this embodiment, the first gate driving circuits 110_1 to 110_n are disposed between the circuit board PCB1 and the detection panel DPL. For example, the first gate driving circuits 110_1 to 110_n are disposed between the circuit board PCB1 and the detection panel DPL by using, for example, methods such as chip on plastic (COP) or a chip on film (COF).

Based on the above, the light detection device in the disclosure may detect the dose of the input light by using the charge generated by at least one second area. The light detection device is not required to sequentially scan all the pixel rows of the light detection device to determine whether the input light is provided by using all the scan lines. In this way, the operation time for AED of the detection device may be shortened.

Although the disclosure has been described with reference to the above embodiments, they are not intended to limit the disclosure. It will be apparent to one of ordinary skill in the art that modifications to the described embodiments may be made without departing from the spirit and the scope of the disclosure. Accordingly, the scope of the disclosure will be defined by the attached claims and their equivalents and not by the above detailed descriptions.

What is claimed is:

1. A light detection device, comprising:
a detection panel configured to convert input light into a charge, wherein the detection panel comprises a plurality of first areas, at least one second area, a plurality of first scan line groups, and at least one second scan line, wherein the first areas are correspondingly coupled to the first scan line groups, wherein the at least one second area is correspondingly coupled to the at least one second scan line;
a plurality of first gate driving circuits correspondingly coupled to the first scan line groups;
a second gate driving circuit coupled to the at least one second scan line; and
a controller coupled to the first gate driving circuits and the second gate driving circuit,
wherein the controller controls the second gate driving circuit during a first period, and detects a dose of the input light by using a charge generated by the at least one second area, and
wherein the controller controls the first gate driving circuits and the second gate driving circuit during a second period, and generates a data image by using a charge generated by the first areas and the at least one second area.

2. The light detection device according to claim 1, wherein during the first period, when the dose is greater than or equal to a critical value, the controller controls the first gate driving circuits and the second gate driving circuit during the second period, so that the charge is generated by the first areas and the at least one second area during the second period.

3. The light detection device according to claim 1, wherein during the first period, when the dose is greater than or equal to a critical value, the controller determines that the input light is provided.

4. The light detection device according to claim 1, wherein during the first period, when the dose is less than a critical value, the controller determines that the input light is not provided.

5. The light detection device according to claim 1, wherein during the first period, the light detection device discharges the charge of the first areas.

6. The light detection device according to claim 1, wherein during the second period, the light detection device stops discharging the charge of the first areas.

7. The light detection device according to claim 1, wherein the second gate driving circuit provides at least one scan signal to the at least one second scan line at the same time during the first period.

8. The light detection device according to claim 1, wherein the second gate driving circuit sequentially provides at least one scan signal to the at least one second scan line during the first period.

9. The light detection device according to claim 1, wherein a plurality of scan lines of the first scan line groups and the at least one second scan line are sequentially driven based on arrangement directions of the first area and the at least one second area.

10. The light detection device according to claim 1, further comprising:
a readout circuit coupled to the first areas, the at least one second area, and the controller to provide the charge generated by at least one of the first areas and the at least one second area to the controller.

11. The light detection device according to claim 1, wherein the controller compensates a partial image corresponding to the at least one second area.

12. The light detection device according to claim 1, wherein the controller compensates a gray level of a partial image corresponding to the at least one second area.

13. The light detection device according to claim 1, wherein the controller compensates a partial image of the at least one second area according to a partial image of the first area adjacent to the at least one second area.

14. The light detection device according to claim 1, wherein the first areas are separated by the at least one second area.

15. The light detection device according to claim 1, wherein
the at least one second area comprises a plurality of second areas, and
the second areas and the first areas are arranged alternately.

16. The light detection device according to claim 1, wherein the input light is an X-ray.

17. The light detection device according to claim 1, further comprising:
a power circuit configured to generate a driving power,
wherein the driving power comprises a power configured to drive the detection panel, the first gate driving circuits, the second gate driving circuit, and the controller.

18. The light detection device according to claim 17, further comprising:
a first circuit board, wherein the controller and the power circuit are disposed on the first circuit board.

19. The light detection device according to claim 18, further comprising:
a second circuit board, wherein the second gate driving circuit is disposed on the second circuit board.

20. The light detection device according to claim 17, wherein the first gate driving circuits are disposed between the first circuit board and the detection panel.
